(19) United States
(12) Patent Application Publication
(10) Pub. No.: US 2013/0071365 A1
(43) Pub. Date: Mar. 21, 2013

(54) INDUCED HEPATOCYTES

(71) Applicant: Atsushi Suzuki, Fukuoka-shi (JP)

(72) Inventor: Atsushi Suzuki, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 13/579,038

(22) PCT Filed: Feb. 16, 2011
(86) PCT No.: PCT/JP2011/053875

§ 371 Date: Nov. 6, 2012

(30) Foreign Application Priority Data
Feb. 16, 2010 (JP) 2010-031718

(51) Int. Cl. C12N 5/071; A61K 48/00; A61K 35/407; C12N 5/07; C12N 15/85

(52) U.S. Cl. CPC C12N 5/067; A61K 35/407
USPC 424/93.21; 435/370; 435/29; 514/1.1; 514/44 R

(57) ABSTRACT

An object of the present invention is to produce hepatic cells from non-hepatic cells that can be obtained less invasively and at low cost. The gene of HNF3α, HNF3β, or HNF3γ and the gene of HNF4α are transduced into non-hepatic cells. The present invention enables the production of induced hepatocytes from somatic cells that are non-hepatic cells, such as skin cells. The use of the induced hepatocytes obtained by the present invention can establish and develop, as general medical treatment, cell transplantation into the liver, artificial livers, and drug response tests, which are the areas that have not been generalized because of the difficulty of procuring hepatocytes.

### TECHNICAL FIELD

The present invention relates to a method of producing hepatic cells from various cells. The present invention is useful in the fields relating to life science, medical treatment, and pharmacology.

### BACKGROUND ART

Hepatocytes are an essential cellular material in each of the following areas: hepatocellular transplantation, which is expected to be a new method of treating liver diseases to replace liver transplantation, which is facing a serious shortage of donor organs; artificial livers, which is on the way of development for the effective treatment of fulminant hepatitis; and drug response tests to assess drug effectiveness and side effects. However, there is a limit to the number of hepatocytes that can be harvested directly from living tissues, and it is difficult to grow hepatocytes ex vivo. For these reasons, the medical application of hepatocytes has yet to go beyond the experimental stage.

Regarding embryonic stem cells (ES cells) and induced pluripotent stem cells (iPS cells), both of which are expected to be new sources of hepatocytes (Patent Document 1), no method of inducing the specific differentiation of these cells into hepatocytes has been found at this time. The risk of tumor formation after the transplantation of those cells, ethical problems, and the like remain as well. Concerning current drug response tests, they use cadaveric hepatocytes or immortalized hepatocytes, which are commercially available from U.S. companies, but these cells have only low-level of functions as hepatocytes and it will be very expensive to continually purchase the cells.

On the other hand, transcriptional regulators for the genes encoding liver-specific metabolic enzymes and serum proteins are in the process of identification. Such transcriptional regulators are called as HNFs (hepatocyte nuclear factors), and, to date, HNF1α, HINF1β (vHNF1), HNF3α, HINF3β, HNF3γ, HNF4α, HNF6, C/EBP family, and GATA family have been identified. Further, studies unrelated to liver development have identified Hlx, Hex, and Prox1 genes as those related to liver development or hepatoblasts. More specifically, it is reported that the expression of HNF3β in human bone marrow-derived mesenchymal stem cells resulted in their differentiation into hepatocyte-like cells (Non-Patent Document 1). Also reported is the induction of the differentiation of human adipose-derived mesenchymal stem cells into hepatocyte-like cells (Non-Patent Document 2). In addition, the present inventors have reported the mechanism of the differentiation of mouse embryonic hepatic stem cells (hepatoblasts) into hepatocytes (Non-Patent Document 3).

### CITATION LIST

### Patent Document

Patent Document 1: WO2007/069666

### Non-Patent Documents

Patent Document 1: Ishii K, Yoshida Y, Akechi Y, Sakabe T, Nishio R, Ikeda R, Terabayashi K, Matsumi Y, Gonda K, Okamoto H, Takubo K, Tajima F, Tsuchiya Hoshikawa Y, Kurimasa A, Umezawa A, Shiota G. Hepatic differentiation of human bone marrow-derived mesenchymal stem cells by tetracycline-regulated hepatocyte nuclear factor 3beta. Hepatology. 2008 August; 48(2): 597-606.
Patent Document 2: Banas A, Teratani T, Yamamoto Y, Tokuhara M, Takeshita F, Quinn G, Okochi H, Ochiya T. Adipose tissue-derived mesenchymal stem cells as a source of human hepatocytes. Hepatology. 2007 July; 46(1): 219-28.
Patent Document 3: Suzuki A., Iwama A., Miyashita H., Nakauchi H., Taniguchi H. Role for growth factors and extracellular matrix in controlling differentiation of prospectively isolated hepatic stem cells. Development 130, 2513-2524, 2003.

### SUMMARY OF INVENTION

### Technical Problem

Hepatocytes are a very useful cell. However, there is no established method to safely and easily obtain hepatocytes that meet the quality and quantity requirements for medical use, and this is a major problem for the medical application of hepatocytes. To solve this problem and achieve the medical use of hepatocytes, it is considered it necessary to develop an unprecedented, innovative new technique for producing hepatocytes from cells that can be obtained less invasively and at low cost.

### Solution to Problem

The present inventor selected twelve transcription factors involved in the hepatocyte differentiation in the course of liver development. The present inventor then incorporated each genes encoding the transcription factors into a retrovirus and transfected mouse embryonic fibroblasts (MEFs) with the retrovirus in various combination. At that time, in the MEFs transduced with a set of HNF4α+HNF3α genes, HNF4α+HNF3β genes, or HNF4α+HNF3γ genes, the induction of the strong expression of albumin, α-fetoprotein (AFP), and E-cadherin was observed. Also, MEFs transduced with HNF4α and HNF3β genes and cultured under appropriate conditions exhibited an epithelial-like cell morphology totally different from the MEFs and were confirmed to express E-cadherin, albumin, and cytokeratin 8-18, as well as α-1-antitrypsin, which is a mid-stage hepatocyte differentiation marker. The present invention has been thus completed.

The present invention provides:

1) A method of producing an induced hepatocyte from a non-hepatic cell, comprising the step of introducing the combination of HNF3α, HNF3β, or HNF3γ and HNF4α (preferably, HNF3γ and HNF4α) into a non-hepatic cell.
2) The method of aspect 1), comprising the step of transducing, into a non-hepatic cell, the gene encoding HNF3α, HNF3β, or HNF3γ and the gene encoding HNF4α (preferably, the gene encoding HNF3γ and the gene encoding HNF4α).
3) The method of aspect 1) or 2), wherein the non-hepatic cell is of human origin.
4) An artificial liver tissue comprising an induced hapatocyte produced by the method of any one of aspects 1) to 3) or the progeny of the induced hapatocyte.
5) A cell derived from a non-hepatic cell and transduced with the gene encoding HNF3α, HNF3β, or HNF3γ and the gene encoding HNF4α (preferably, the gene encoding HNF3γ and the gene encoding HNF4α).
6) A cell induced from a non-hepatic cell transduced with the gene encoding HNF3α, HNF3β, or HNF3γ and the gene encoding HNF4α (preferably, the gene encoding HNF3γ and the gene encoding HNF4α), wherein the induced cell being E-cadherin-positive and capable of expressing albumin, cytokeratin-8, cytokeratin-18, or α-1-antitrypsin.
7) A set of factors, such as a kit, comprising the combination of HNF3α, HNF3β, or HNF3γ and HNF4α (preferably, HNF3γ and HNF4α), the set of factors being intended for use in a method of producing a hepatic cell from a non-hepatic cell.
8) A set of genes, such as a kit, comprising the combination of the gene encoding HNF3α, HNF3β, or HNF3γ and the gene encoding HNF4α (preferably, the gene encoding HNF3γ and the gene encoding HNF4α), the set of genes being intended for use in a method of producing a hepatic cell from a non-hepatic cell.
9) A method of producing an induced hepatic cell, comprising the step of culturing, in the presence of a growth factor (optionally, on an extracellular matrix), a non-hepatic cell transduced with the gene encoding HNF3α, HNF3β, or HNF3γ and the gene encoding HNF4α (preferably, the gene encoding HNF3γ and the gene encoding HNF4α).
10) A method of transplanting a cell into liver tissue, comprising the step of transplanting the cell of aspect 5).
11) A method of treating diseases, comprising a step using the cell of aspect 5).
12) A method of assessing drug responsiveness, comprising a step using the cell of aspect 5).

### Advantageous Effects of Invention

The present invention enables the production of hepatic cells from somatic cells that are non-hepatic cells, such as skin cells. The use of hepatic cells obtained by the present invention can establish and develop, as general medical treatment, hepatocellular transplantation, artificial livers, and drug responsiveness tests, which are the areas that have not been generalized because of the difficulty of procuring hepatocytes.

### BRIEF DESCRIPTION OF DRAWINGS

FIG. 1 (Gene expression-inducing effect of all but any one of twelve factors) FIG. 1 is a graph showing the gene expression-inducing effect of the selected twelve transcription factors involved in hepatocyte differentiation. In the MEFs transduced with all the twelve factors, the gene expression of albumin, α-fetoprotein, and E-cadherin was observed. When each of the sets of the genes encoding all but any one of the twelve factors was transduced into MEFs, the MEF group lacking HNF4α or HNF6 showed decreased expression of albumin gene, and the group lacking HNF4α or HNF1β showed decreased expression of α-fetoprotein gene. However, there was no group showing the marked decrease in E-cadherin gene expression.

FIG. 2 (Gene expression-inducing effect of one factor+HNF4α) FIG. 2 is a graph showing the effect of the simultaneous transduction of HNF4α and another factor. When a set of HNF4α+HNF3α genes, HNF4α+HNF3β genes, or HNF4α+HNF3γ genes was transduced, the strong induction of expression of albumin, α-fetoprotein, and E-cadherin was observed.

FIG. 3 (Growth of epithelial-like cells after transduction of HNF4α and HNF3β genes) FIG. 3 is a photograph of induced epithelial-like cells. When the MEFs transduced with HNF4α and HNF3β genes were transferred into a type I collagen-coated dish and cultured in a medium for culture of hepatic stem cells, epithelial-like cells totally different in cell morphology from the MEFs emerged.

FIG. 4 (Expression of E-cadherin, albumin, CK8-18, and α-1-antitrypsin in epithelial-like cells that emerged by transduction of HNF4α and HNF3β) FIG. 4 is a photograph of fluorescent stained image of the epithelial-like cells obtained by the transduction of HNF4α and HNF3β genes and stained fluorescently. All the epithelial-like cells induced from the MEFs were E-cadherin-positive and E-cadherin was localized in cell-adhesion domains. In addition, many of the E-cadherin-positive cells expressed albumin and cytokeratin 8-18 genes. The expression of α-1-antitrypsin was also observed (the graph).

FIG. 5 (Reconstruction of liver tissues by MEF-derived epithelial-like cells) FIG. 5 is a photograph at one month after the transplantation of the epithelial-like cells induced from the MEFs into the liver of an FAH knockout mouse. The photograph reveals that the epithelial-like cells grown into FAH-positive mature hepatocytes were engrafted into the mouse liver tissues and reconstructed them. This result shows that the epithelial-like cells induced from the MEFs by the transduction of HNF4α and HNF3β were liver epithelial cells having the ability to reconstruct liver tissues. Into a control mouse, unmodified MEFs were transplanted.

FIG. 6A shows the nucleotide sequences of the (mouse) gene encoding each of HNF4α, HNF3α, HNF3β, and HNF3γ.

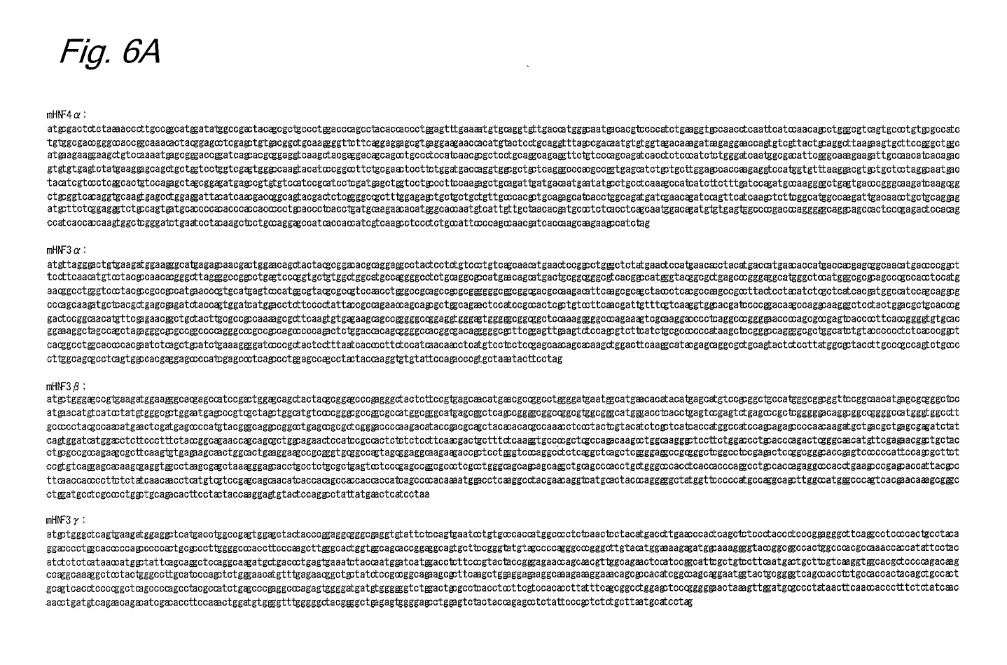

FIG. 6B shows the nucleotide sequences of the (human) gene encoding each of HNF4α, HNF3α, HNF3β, and HNF3γ.

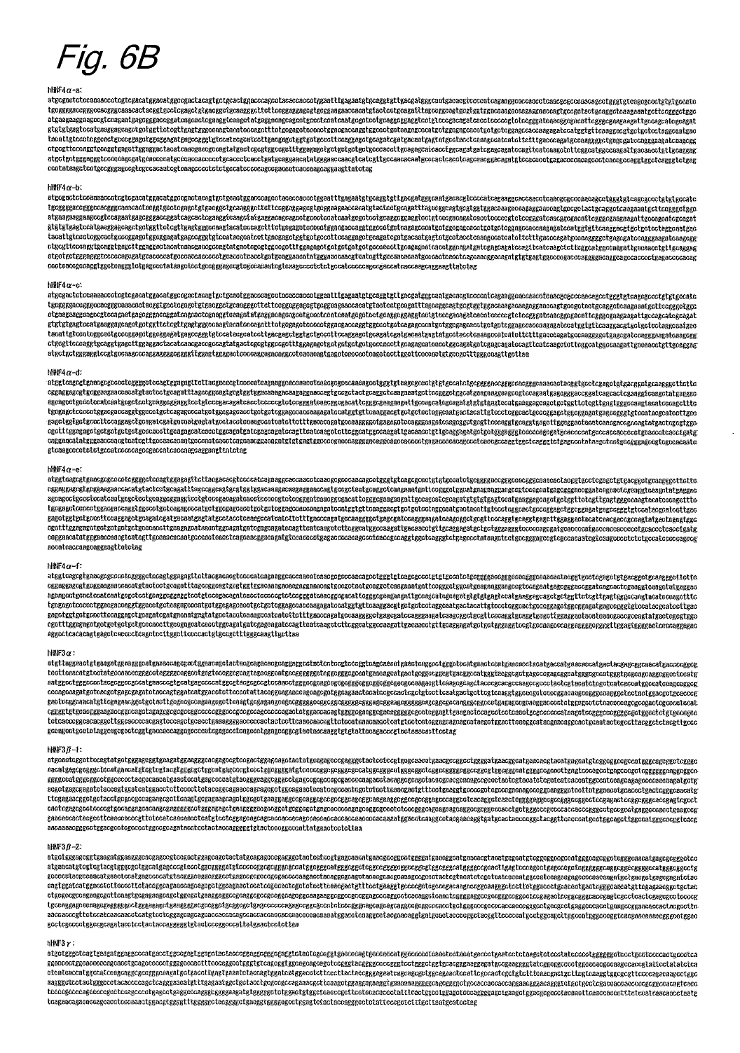

FIG. 7 shows the (mouse or human) amino acid sequence of each of HNF4α, HNF3α, HNF3β, and HNF3γ.

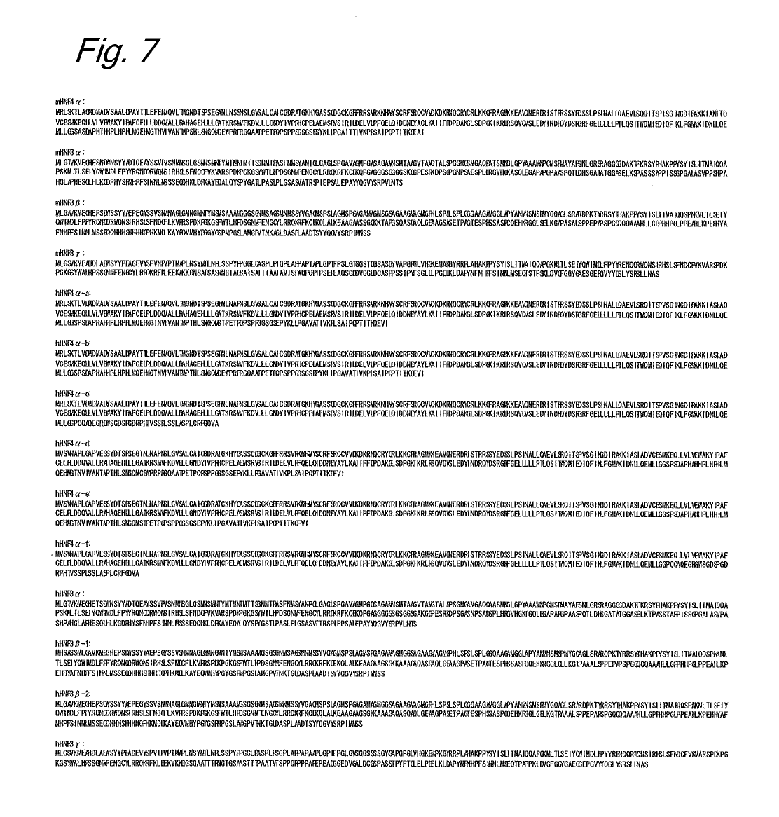

FIG. 8 (Gene expression-inducing effect of transduction of HNF4α and HNF3γ in human skin-derived fibroblasts) FIG. 8 is a graph showing the gene expression-inducing effect of the simultaneous transduction of HNF4α and HNF3γ into human skin-derived fibroblasts. As in the case of the mouse-derived fibroblasts, the induction of the strong expression of albumin, α-fetoprotein, and E-cadherin was observed.

### DESCRIPTION OF EMBODIMENTS

The present invention can produce hepatic cells by transducing a set of specific factors into various cells.

[Sets of Factors for Producing Hepatic Cells]

The sets of factors for producing hepatic cells according to the present invention were selected from the group consisting of the twelve factors that the present inventor deems are expressed in hepatoblasts (hepatic stem cells/progenitor cells) or endodermal cells and related to or possibly related to hepatocyte differentiation. More specifically, the twelve factors are Hex, GATA4, GATA6, Tbx3, C/EBPα, HNF1α, HNF1β, HNF3α, HNF3β, HNF3γ, HNF4α, and HNF6.

The set of factors according to the present invention includes at least HNF4α. A preferred embodiment is characterized in that the combination of HNF3α, HNF3β, or HNF3γ and HNF4α is included, and a especially preferred embodiment includes HNF4α and HNF3β.

Mammals including humans have all of these factors in common. The factors used in the present invention may be derived from any mammal, unless otherwise specified. In the present invention, examples of the factors that can be used include those derived from mouse, rat, cow, sheep, horse, monkey, or human.

The factors used in the present invention are not limited to specific isoforms. Humans have at least six isoforms of HNF4α and two isoforms of HNF3β, and any of the isoforms can be used in the present invention.

The table below shows the factors particularly relevant to the present invention and the NCBI accession numbers of the nucleotide sequences of the mouse and human genes encoding the factors.

TABLE 1

| | | NCBI | NCBI |
|---|---|---|---|
| | | accession | accession |
| Name | Features | No. (mouse) | No. (human) |
| HNF3α | Transcription factor having a forkhead DNA- | NM_008259 | NM_004496 |
| | binding domain. | | |
| | Involved in the development of endoderm, | | |
| | liver, and pancreas. | | |
| HNF3β | Transcription factor having a forkhead DNA- | NM_010446 | NM_021784 |
| | binding domain. | | NM_153675 |
| | Involved in the development of endoderm, | | |
| | liver, and pancreas. | | |
| HNF3γ | Transcription factor having a forkhead DNA- | NM_008260 | NM_004497 |
| | binding domain. | | |
| | Involved in the development of endoderm, | | |
| | liver, and pancreas. | | |
| HNF4α | Transcription factor belonging to a nuclear | NM_008261 | NM_178849 |
| | receptor family. | | NM_000457 |
| | Involved in hepatocyte differentiation and cell | | NM_178850 |
| | morphology control. | | NM_175914 |
| | | | NM_001030003 |
| | | | NM_001030004 |

The Sequence Listing included in this application shows nucleotide sequences of murine HNF4α gene, HNF3α gene, HNF3β gene, and HNF3γ gene of SEQ ID NOs: 1 to 4 in that order, respectively. The Sequence Listing also shows nucleotide sequences of six isoforms of human HNF4α gene as SEQ ID NOs: 5 to 10, respectively; human HNF3α gene as SEQ ID NO: 11; the two isoforms of human HNF3β gene as SEQ ID NOs: 12 and 13, respectively; and human HNF3γ gene as SEQ ID NO: 14. The amino acid sequences of the respective growth factors can be deduced from the gene sequences. The Sequence Listing included in this application shows amino acid sequences of murine HNF4α, HNF3α, HNF3β, and HNF3γ as SEQ ID NOs: 15 to 18 in that order, respectively. The Sequence Listing also shows the amino acid sequences of the six isoforms of human HNF4α (transcript variants 1 to 6) as SEQ ID NOs: 19 to 24, respectively; human HNF3α as SEQ ID NO: 25; the two isoforms of human HNF3β as SEQ ID NOs: 26 and 27, respectively; and human HNF3γ as SEQ ID NO: 28.

The study of the present inventor reveals that although gene expression was induced by HNF4α+HNF3α or HNF4α+HNF3β in an experiment using mouse-derived cells, there was the tendency that the combination of HNF4α and HNF3γ exhibited the greatest gene expression-inducing effect. Likewise, it was confirmed that gene expression had been induced by HNF4α+HNF3α or HNF4α+HNF3β in human-derived cells; however, as in the case of the mouse-derived cells, there was the tendency that the combination of HNF4α and HNF3γ was the most effective. Additionally, in the experiment using the mouse-derived cells, whichever combination of the factors shown above was transduced, the resultant cells were not found to be significantly different, despite the differences in albumin secreting ability, gene expression profiles, and the like. Further, the transcript variants 1 to 5 of HNF4α were tested using human-derived cells and the results appear that the transcript variants 1 to 3, especially, the transcript variant 3 was effective.

The scope of the present invention includes use of the factors described above and that of the genes encoding the factors, as well as use of variants thereof. More specifically, the certain factor as used in the present invention includes, unless otherwise specified, (a) a protein consisting of the amino acid sequence of specified SEQ ID No. shown in the Sequence Listing; (b) a protein consisting of an amino acid sequence having substitution, deletion, insertion, and/or addition of one or more amino acids in the amino acid sequence referred to in (a) and which has the same function as that of the factor; and (c) a protein consisting of an amino acid sequence having at least 90% (preferably 95%, more preferably 98%) identity with the amino acid sequence referred to in (a) and which has the same function as that of the factor. The certain factor-encoding gene as used in the present invention includes, unless otherwise specified, (d) a gene consisting of the nucleotide sequence of specified SEQ ID No. in the Sequence Listing; (e) a gene that hybridizes, under stringent conditions, with a polynucleotide of a sequence complementary to the nucleotide sequence referred to in (d) and which encodes a protein having the same function as that of the factor; (f) a gene consisting of a sequence having at least 90% (preferably 95%, more preferably 98%) identity with the nucleotide sequence referred to in (d) and which encodes a protein having the same function as that of the factor; and a gene encoding any one of the proteins (a) to (c).

The methods to obtain these variants are well known by a person skilled in the art. It is to be noted that the term “stringent conditions” as used herein means conditions of 6M urea, 0.4% SDS, and 0.5×SSC or hybridization conditions equivalent thereto. If needed, more stringent conditions such as those of 6M urea, 0.4% SDS, and 0.1×SSC or hybridization conditions equivalent thereto may be applied to the present invention. Under the respective conditions, the temperature may be about 40° C. or higher, and if more stringent conditions are needed, the temperature may be, for example, about 50° C. or even about 65° C. The homology between amino acid sequences or between nucleotide sequences can be determined using the BLAST algorithm, which was developed by Karlin and Altschul (Proc. Natl. Acad. Sci. USA 87: 2264-2268, 1990; Proc Natl Acad Sci USA 90: 5873, 1993). The BLAST algorithm-based programs, each of which is called BLASTN or BLASTX, have also been developed (Altschul S F, et al: J Mol Biol 215: 403, 1990). If a nucleotide sequence is analyzed using BLASTN, the appropriate parameters may be, for example, score=100 and wordlength=12. If an amino acid sequence is analyzed using BLASTX, the appropriate parameters may be, for example, score=50 and wordlength=3. If the BLAST or Gapped BLAST program is used, its default parameters are used. The concrete techniques of these analysis methods are known (http://www.ncbi.nlm.nih.gov/).

The factor set according to the present invention can include not only the factors described above but also the other factors. The table below shows such other factors and the NCBI accession numbers of the nucleotide sequences of the mouse and human genes encoding the factors.

TABLE 2

| | | NCBI | NCBI |
|---|---|---|---|
| | | accession | accession |
| Name | Features | No. (mouse) | No. (human) |
| Hex | Homeodomain-type transcription factor. | NM_008245 | NM_002729 |
| | Involved in hepatoblast migration and | | |
| | differentiation at the time of liver development. | | |
| GATA4 | Transcription factor belonging to the GATA | NM_008092 | NM_002052 |
| | transcription factor family. | | |
| | Involved in endoderm development. | | |
| GATA6 | Transcription factor belonging to the GATA | NM_010258 | NM_005257 |
| | transcription factor family. | | |
| | Involved in liver development. | | |
| Tbx3 | Transcription factor belonging to the T-box | NM_198052 | NM_005996 |
| | transcription factor family. | | |
| | Involved in the growth of hepatoblasts and | | |
| | their differentiation into hepatocytes at the time | | |
| | of liver development. | | |
| C/EBPα | Transcription factor having a basic amino acid | NM_007678 | NM_004364 |
| | domain and leucine zipper. | | |
| | Involved in hepatocyte differentiation at the | | |
| | time of liver development. | | |
| HNF1α | Homeodomain-type transcription factor. | NM_009327 | NM_000545 |
| | Involved in the development of endoderm, | | |
| | liver, and pancreas. | | |
| HNF1β | Homeodomain-type transcription factor. | NM_009330 | NM_000458 |
| | Involved in the development of endoderm, | | |
| | liver, and pancreas and the differentiation and | | |
| | maturation of bile duct cells. | | |
| HNF6 | Homeodomain-type transcription factor. | NM_008262 | NM_004498 |
| | Involved in hepatoblast differentiation into | | |
| | hepatocytes and the differentiation and | | |
| | maturation of bile duct cells, at the time of liver | | |
| | development. | | |

Tbx3 is a transcription factor identified and reported by the present inventors (Suzuki A., Sekiya S., Buscher D., Izpisua Belmonte J. C., Taniguchi H. Tbx3 controls the fate of hepatic progenitor cells in liver development by suppressing p19ARF expression. Development 135, 1589-1595, 2008).

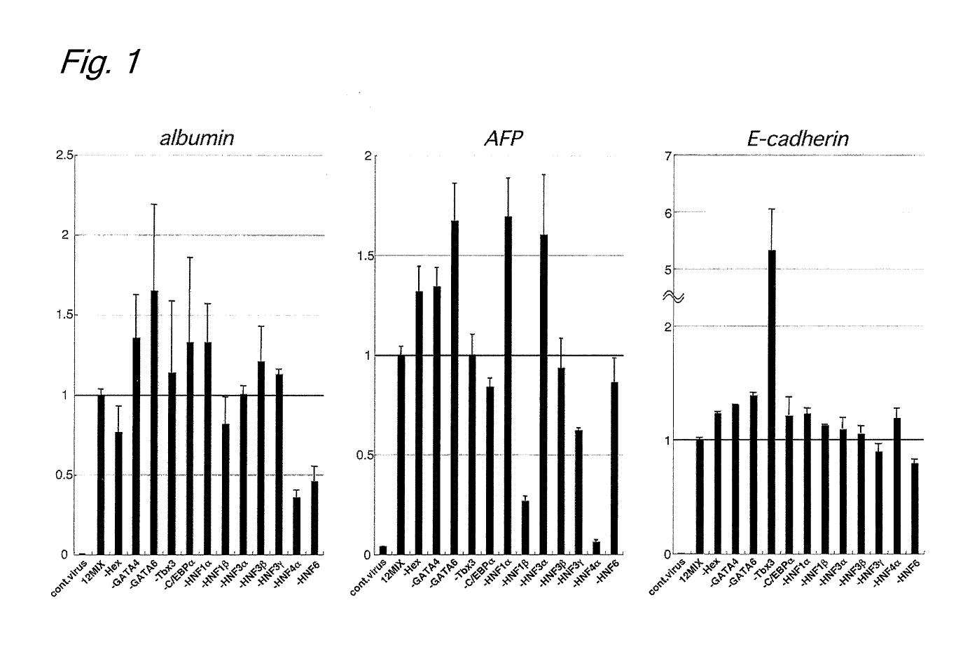

In view of the results of the Example shown herein (FIG. 1), it is considered that the impacts of HNF1β and HNF6 need to be investigated as well.

A set of factors for producing hepatic cells of the present invention may include not only the factors described above but also one or more other factors. Such one or more other factors can be selected by, for example, transducing the genes that encode various candidate factors into somatic cells that express one or more factors selected from the group consisting of HNF3α, HNF3β, HNF3γ, and HNF4α and choosing cells that can induce hapatic cells at a higher rate.

The factor used in the present invention may be a protein itself produced from the gene encoding the factor or may be in the form of a fusion protein with another protein, peptide, or the like. For example, the factor may be a fusion protein with the green fluorescent protein (GFP) or a fusion protein with a peptide such as a histidine tag. Alternatively, the factor may be a fusion protein with a PTD (protein transduction domain) peptide. The methods of preparing such fusion proteins are well known by a person skilled in the art.

[Transduction Means]

A method of preparing hepatic cells according to the present invention includes a step of transducing specific factors into target cells, but the specific means to carry out this step is not particularly limited. Each of the factors is originally considered to be transcribed and translated from a gene within a cell and function as a protein (as a transcription factor). Thus, an intended advantageous effect can be achieved by direct introduction of the factors into cells or the transduction of the genes encoding the factors into cells in a physicochemical manner or by use of a virus vector.

A typical example of the means of introducing the factors directly into cells is a method by binding the factor to a transmembrane PTD peptide (e.g., AntP, HIV/TAT, HSV/VP-22). This method is superior in that it can be applied to individuals in vivo and enables the factors to be introduced into almost all cells including those into which genes could not have been easily transfected by existing transfection techniques. The method is also superior in that the introduction of the factors needs only the addition of the fusion protein to a culture environment, avoiding the complicated manipulations for gene transduction into cells. A method of preparing a fusion protein of a target protein and a PTD, the conditions for transducing a fusion protein into target cells, and the like are well-known to the person skilled in the art.

Examples of the means of transducing genes into cells physicochemically include calcium phosphate method/lipofection method, electroporation, ultrasonic gene transduction, gene gun-mediated transduction, and recombinant immunogene method. All of these methods are well known to a person skilled in the art.

Examples of the virus vector used for transducing genes into cells include retrovirus vector, lentivirus vector, Sendai virus vector, and helper-dependent adenovirus vector. The method for preparing a vector that carries the gene encoding a factor, the method for transfecting cells with such a vector, and the like are well known to a person skilled in the art. From the viewpoint of the capability of transducing genes efficiently even into cells lacking high growth ability, a method using a lentivirus vector is considered to be preferable.

A person skilled in the art can confirm appropriately whether intended genes have been transduced. For example, on a vector, a marker gene (a fluorescent protein-encoding gene, a drug-resistant gene, etc.) is linked, via an IRES (internal ribosome entry site), to the downstream of a gene to be transduced, thereby allowing the marker to be expressed simultaneously with the intended gene product; in this way, the cells transduced with the intended gene can be selected or visualized. Whether two intended genes simultaneously transduced into a cell can be confirmed by preparing a construct where genes are linked to different marker genes from each other; for example, one of the intended genes is joined to GFP (green) and the other to RFP1 (red). Further, if the cells trancduced with genes can be cloned as a cell line, the presence or absence of the transduced genes can be confirmed by PCR or Southern blotting in the whole cells.

[Target Cells for Factor Transduction]

In the present invention, the cells subjected to factor transduction are non-hepatic cells. The “non-hepatic cells” as used in the present invention, unless otherwise specified, mean the non-hepatic cells that will be described below.

The target cells for factor transduction may be derived from any animals. Cells derived from, for example, mouse, rat, cattle, sheep, horse, monkey, or human can be exemplified as a target cell in the present invention.

The target cells for factor transduction may be embryonic stem cells (ES cells) or induced pluripotent stem cells (iPS cells), or other somatic cells. Embryonic stem cells (ES cells) are a stem cell line produced from inner cell mass, which belongs to a part of embryos at blastocyst stage, which is an early developmental stage of an animal. Induced pluripotent stem cells (iPS cells) are a cell obtained by the transduction of genes for specific factors into somatic cells. As in the case of ES cells, iPS cells have pluripotency which enables the cells to differentiate into numerous types of cells and self-renewal ability which enables the cells to keep the pluripotency of the cell after their division growth.

The somatic cells include the following cells: stem cells which have the ability to differentiate into several types of cells having different functions (e.g., mesenchymal stem cells); and differentiated cells that are specialized for certain functions and do not grow into other cells (e.g., fibroblasts, adult skin cells).

The target cells for factor transduction may be germline cells, germline cell-derived cells, or amniotic cells (including placental cells and the like).

In the production of hepatic cells using the present invention, the target cells used herein are not particularly limited and may be fetal cells or adult-derived mature cells.

If a gene is transduced by a retrovirus, it may be preferred, in some cases, that cells capable of growing vigorously are selected. Examples of the cells include fetus-derived fibroblasts and adult bone marrow-derived mesenchymal stem cells.

From the viewpoint of the use of the hepatic cells obtained by the present invention for the treatment of human diseases, the target cells used herein are preferably a cell of human (adult) origin, such as fibroblasts obtained from skin cells. In some cases, the target cells may be preferable autologous cells separated from patients themselves or isogenic cells separated from isogenic humans.

[Hepatic Cells, Induced Hepatocytes]

The “hepatic cells” as used in the present invention include, unless otherwise specified, fetal or adult cells that consist of the liver, fetal or adult hepatic progenitor cells (also referred to as hepatic stem cells), and fetal or adult cells partially differentiated into hepatocytes. The cells that consist of the liver include hepatic parenchymal cells (such as hepatocytes) and hepatic non-parenchymal cells (e.g., sinusoidal endothelial cells, Kupffer cells, stellate cells, pit cells, biliary epithelial cells).

The cells induced from non-hepatic cells into cells having the function of hepatic cells by the present invention can be referred to as induced hepatocytes (1Heps), in order to distinguish them from existing hepatic cells. Induced hepatocytes include hepatocyte-like cells as well as hepatic progenitor cell-like cells or cells which are like cells partially differentiated into hepatocytes.

A person skilled in the art can determine whether cells transduced with a certain factor have grown into induced hepatocytes, by morphological observation and/or based on the following indexes: the presence or absence of the expression of albumin, which is a hepatocyte differentiation marker; the presence or absence of the expression of α-1-antitrypsin, which is a mid-stage hepatocyte differentiation marker; and the presence or absence of glycogen storage capacity. Optionally, α-fetoprotein expression may be confirmed. In addition, in some specific hepatocytes, the determination may be made based on the following indexes: the presence or absence of cytokeratin 8 and/or cytokeratin 18 (CK 8/18) expression; the presence or absence of the expression of CYP7A1 (cholesterol 7a-hydroxylase), which catalyzes the first-step reaction in the biosynthetic pathway from cholesterol to bile acid; and the presence or absence of the expression of E-cadherin, which is a major adhesion molecule for epithelial cells.

[Induction Method]

In the present invention, cells transduced with genes for HNF3α, HNF3β, or HNF3γ and HNF4α are cultured, if needed, in a standard medium (e.g., SCM (see References 1 to 4 shown below)) for several hours to several weeks (e.g., about two weeks), and then cultured under appropriate conditions such that the cells are induced to have the function of hepatic cells. The appropriate culture conditions include culture conditions where a growth factor is contained in the environment for maintaining the cells and an extracellular matrix is optionally contained in the environment. As the medium, the standard medium described in Reference 2 shown below can be preferably used.

Examples of the growth factors that can be contained in the culture condition include cytokines such as hepatocyte growth factor (HGF) and epidermal growth factor (EGF). A medium containing HGF and EGF is preferred.

The concentration of the growth factor in the medium is not particularly limited as long as the induction is possible, and the concentration also varies depending on the type of the growth factor used. The concentration of EGF ranges between 10 and 40 ng/mL, preferably about 20 ng/mL, and the concentration of HGF ranges between 10 and 50 ng/mL, preferably between 20 and 40 ng/mL.

The origin of growth factor is not particularly limited and may be a human recombinant growth factor.

The extracellular matrix is exemplified as, for example, collagen (e.g., type I collagen, type IV collagen) or laminin. Type I collagen is preferably used.

The extracellular matrix is preferably used as a coating agent for a culture substrate. A culture vessel can be coated with the extracellular matrix by a method commonly used in the art for each of the type of the matrix.

In the case of use of human adult-derived cells as the cells subjected to introduction or transduction of the factors in the present invention, human adult-derived cells have lower growth ability than mouse-derived cells in many cases. However, the induction medium used in the Examples of the present invention can be applied to human-derived cells as well, partially because the medium contains human recombinant growth factors. In the medical application of the induced hepatocytes obtained, a culture system that is serum-free and which excludes substances derived from other living creatures than a human is preferably used to induce cells.

In the culture for induction, examples of the culture vessel that can be used include, but are also not particularly limited to, flask, dish, multidish, microwell plate, culture bag, and roller bottle. As other culture conditions, various known conditions for culturing hepatic cells can be applied. For example, the culture temperature ranges between 30 and 40° C., preferably 37° C. The concentration of CO₂ ranges between, for example, 1 and 10%, preferably between 2 and 5%.

The culture for induction can be continued for generally one day to several weeks, preferably one to three weeks, for example, two weeks. By confirming the presence or absence of the expression of the hepatocyte differentiation markers described above and conducting morphological observation in an appropriate phase of the culture, they can help determining the end of the culture or the next phase such as the phase of the transition to the passage culture of cells in an incubator that allows three-dimensional culture for the construction of hepatic tissues, or the transition to the administration to individuals.

According to the study of the present inventors, the cells obtained by the transduction of HNF4α and HNF3β into MEFs are cultured under appropriate conditionsto form an epithelial-like cell population that expressed hepatocyte markers and which continued to grow, and the cell population could be subjected to the subsequent passage culture and cryopreservation. It is considered to show that differentiated cells and less differentiated progenitor cells coexist in the obtained cell population.

[Intended Uses]

The intended uses of the cells produced by the inventive method are not particularly limited, and the cells can be used for any current test and study using cells, treatment of diseases using hepatic cells, and the like. For example, the induced hepatocytes obtained by the present invention or their progeny can consist of artificial hepatic tissues in vitro or in vivo. Cell transplantation into the liver can be achieved by use of the administration of the obtained induced hepatocytes or their progeny to patients by an appropriate method. Such a transplantation technique is considered to be effective for the treatment of various liver diseases including those treatable by partial hepatectomy and/or liver transplantation (e.g., diseases or conditions such as cirrhosis, primary biliary cirrhosis, viral hepatitis, alcoholic hepatitis, autoimmune hepatitis, liver cancer, hepatocellular cancer, cholangiocellular cancer, metastatic liver cancer, liver abscess, and congenital dysmetabolic syndromes such as hereditary hypertyrosinemia type I (fumarylacetoacetate hydrolase deficiency, hepatorenal tyrosinemia). The present invention provides a method of treating liver diseases using the combination of HNF3α, HNF3β, or HNF3γ and HNF4α.

### Example 1

### Method

Cell Culture

Mouse embryonic fibroblasts (MEFs) were obtained from a mouse fetus (C57BL/6 E13.5) and cultured in a DME medium (Dulbecco's modified Eagle medium) supplemented with 10% fetal bovine serum (FBS), L-glutamine (2 mmol/L), and penicillin/streptomycin. Retrovirus-mediated gene transduction into the growing MEFs was repeated five times.

One day after the final transduction, the medium was replaced by an SCM (standard culture medium; see References 1 to 4). The SCM was a medium obtained by the addition of the following substances to a 1:1 mixture of a DMEM and F-12 (Nacalai Tesque):

10% FBS

insulin (1 μg/mL) (Wako, Tokyo, Japan)

dexamethasone (1×10⁷ mol/L) (Sigma)

nicotinamide (10 mmol/L) (Sigma)

L-glutamine (2 mmol/L)

β-mercaptoethanol (50 μmol/L) (Sigma)

penicillin/streptomycin

After a two-week culture in the SCM medium, the cells were transferred into a type I collagen-coated dish (Iwaki Glass, Tokyo, Japan) and cultured in a medium supplemented with 20 ng/mL human recombinant hepatocyte growth factor (HGF) (Sigma) and 20 ng/mL epidermal growth factor (EGF) (Sigma).

Gene Expression Analysis

RNeasy Mini Kit (QIAGEN, Tokyo, Japan) was used to obtain total RNA in accordance with the manual attached to the kit. TaqMan Universal PCR Master Mix (Applied Biosystems, Japan) and Applied Biosystems 7300 Realtime PCR System (Applied Biosystems) were used to perform quantitative PCR (qPCR). The information on the PCR primers and probes is shown in References 1 to 3, except those for E-cadherin (TaqMan Gene Expression Assay ID: Mm00486909 g1) (Applied Biosystems).

Retrovirus Production

The retrovirus vector pGCsam (murine stem cell virus, MSCV) (see Reference 1) was used. The following respective mouse-derived genes encoding the twelve factors were subcloned into vectors:

TABLE 3

| | | | Corresponding |
|---|---|---|---|
| | | NCBI Accession | Amino Acid |
| | | No. | Sequence |
| | | (SEQ ID No. in | (SEQ ID No. in |
| Gene | Features | Sequence Listing) | Sequence Listing) |
| Hex | Homeodomain-type transcription factor. | NM_008245 | |
| | Involved in hepatoblast migration and differentiation at | | |
| | the time of liver development. | | |
| GATA4 | Transcription factor belonging to the GATA | NM_008092 | |
| | transcription factor family. | | |
| | Involved in endoderm development. | | |
| GATA6 | Transcription factor belonging to the GATA | NM_010258 | |
| | transcription factor family. | | |
| | Involved in liver development. | | |
| Tbx3 | Transcription factor belonging to the T-box transcription | NM_198052 | |
| | factor family. | | |
| | Involved in the growth of hepatoblasts and their | | |
| | differentiation into hepatocytes at the time of liver | | |
| | development. | | |
| C/EBPα | Transcription factor having a basic amino acid domain | NM_007678 | |
| | and leucine zipper. | | |
| | Involved in hepatocyte differentiation at the time of liver | | |
| | development. | | |
| HNF1α | Homeodomain-type transcription factor. | NM_009327 | |
| | Involved in the development of endoderm, liver, and | | |
| | pancreas. | | |
| HNF1β | Homeodomain-type transcription factor. | NM_009330 | |
| | Involved in the development of endoderm, liver, and | | |
| | pancreas and the differentiation and maturation of bile | | |
| | duct cells. | | |
| HNF3α | Transcription factor having a forkhead DNA-binding | NM_008259 | (SEQ ID NO: 16) |
| | domain. | (SEQ ID NO: 2) | |
| | Involved in the development of endoderm, liver, and | | |
| | pancreas. | | |
| HNF3β | Transcription factor having a forkhead DNA-binding | NM_010446 | (SEQ ID NO: 17) |
| | domain. | (SEQ ID NO: 3) | |
| | Involved in the development of endoderm, liver, and | | |
| | pancreas. | | |
| HNF3γ | Transcription factor having a forkhead DNA-binding | NM_008260 | (SEQ ID NO: 18) |
| | domain. | (SEQ ID NO: 4) | |
| | Involved in the development of endoderm, liver, and | | |
| | pancreas. | | |
| HNF4α | Transcription factor belonging to a nuclear receptor | NM_008261 | (SEQ ID NO: 15) |
| | family. | (SEQ ID NO: 1) | |
| | Involved in hepatocyte differentiation and cell | | |
| | morphology control. | | |
| HNF6 | Homeodomain-type transcription factor. | NM_008262 | |
| | Involved in hepatoblast differentiation into hepatocytes | | |
| | and the differentiation and maturation of bile duct cells, | | |
| | at the time of liver development. | | |

For production of a retrovirus, 293 cells containing the gag and pol genes but not containing the env gene was provided together with a mixture of a plasmid DNA and a VSV-G env expression plasmid (pCMV-VSV-G) (provided by H. Miyoshi) along with polyethylenimine (PEI) (Polysciences Inc., Warrington, Pa.). Then, the supernatant was harvested from the thus obtained transfected cells and centrifuged at 9000 g at 4° C. for 12 hours to concentrate the virus.

For the transfection, 50 μL of the 140-fold concentrated virus was used in each well of a 12-well culture plate.

Immunostaining

Tissues and the cultured cells were fixed and incubated with the following primary antibodies: anti-albumin antibody (Biogenesis, Poole, UK), anti-E-cadherin antibody (BD Biosciences), anti-cytokeratin (CK) 8-18 antibody (Progen, Heidelberg, Germany; both CK8 and CK18 molecules can be detected), and anti-fumarylacetoacetate hydrolase (FAH) antibody (provided by R. M. Tanguay). After washing the tissues and cells, an HRP (horse radish peroxidase)-labeled secondary antibody (1:500; Dako) or Alexa 488- and/or Alexa 555-labeled secondary antibodies (1:200; Molecular Probes, Eugene, Oreg.) were added to the tissues and cells, which were then incubated with 4′,6-diamino-2-phenylindole (DAPI).

Cell Transplantation

Epithelial-like cells induced from the MEFs (or intact MEFs as a control) were treated with trypsin, washed, and suspended in 200 μl SCM, and the suspension was injected via the portal vein to thereby transplant the cells into the liver of an FAH-deficient mouse (FAH−/−; see Reference 4) (1×10⁷ cells/mouse). FAH−/− mice are known as a model for hereditary hypertyrosinemia type I and generally maintained by supplementation with water containing 7.5 mg/L of 2-(2-nitro-4-trifluoromethylbenzoyl)-1,3-cyclohexanedione (NTBC) (Swedish Orphan International). However, the supplementation was ceased after this cell transplantation.

2. Results

Identification of Reprogramming Factors that Change Fibroblasts into Hepatocytes

The genes encoding the twelve transcription factors related to hepatocyte differentiation in the course of liver development were incorporated into the retrovirus, which was then transfected into the mouse embryonic fibroblasts (MEFs).

Two weeks after the transfection, the gene expression of albumin and α-fetoprotein (which are expressed in hepatocytes and hepatic progenitor cells), and the gene expression of E-cadherin were (the epithelial cell marker) observed in the MEFs transduced with all the twelve factors (FIG. 1). Then, in order to select from the twelve factors the gene(s) having the reprogramming function to change the MEFs into hepatocytes, the MEFs were transfected with each of the sets of the eleven genes encoding all but any one of the twelve factors. As a result, the MEF group lacking HNF4α or HNF6 showed decreased expression of albumin gene, and the MEF group lacking HNF4α or HNF1β showed decreased expression of α-fetoprotein gene. Meanwhile, none of the groups lacking any one of the twelve factors showed the marked decrease in E-cadherin gene expression (FIG. 1).

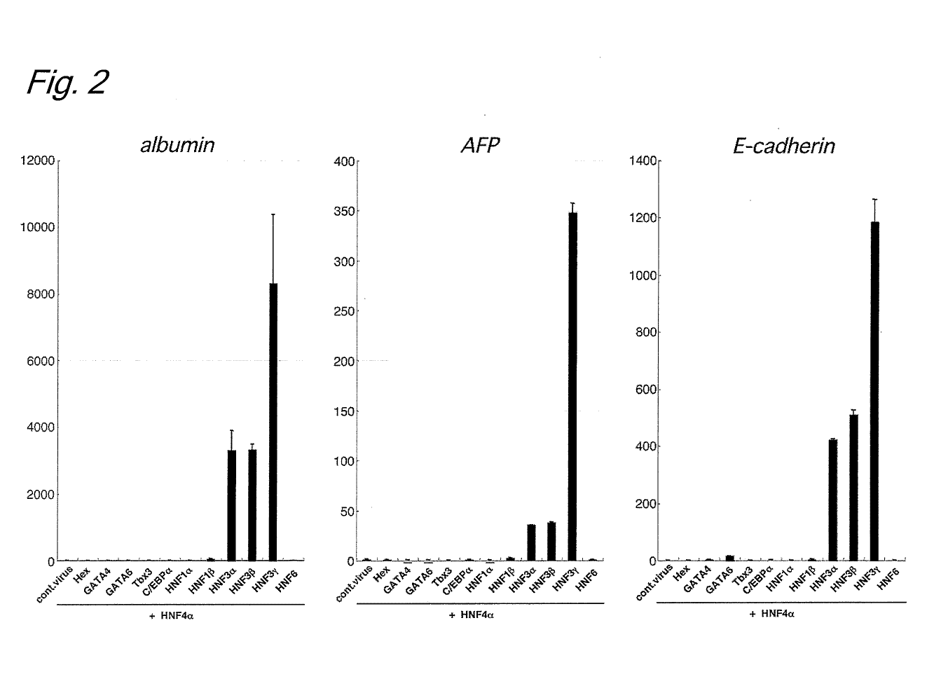

With the attention focused on HNF4α, which suggest involving in the expression of both of albumin and α-fetoprotein genes, the present inventor made an experiment to transfect the MEFs simultaneously with HNF4α and another factor. As a result, when a set of genes of HNF4α+HNF3α, HNF4α+HNF3β, or HNF4α+HNF3γ was transduced, the strong induction of the expression of albumin, α-fetoprotein, and E-cadherin was observed (FIG. 2).

Emergence of Hepatic Epithelial Cells Caused by Reprogramming of Fibroblasts

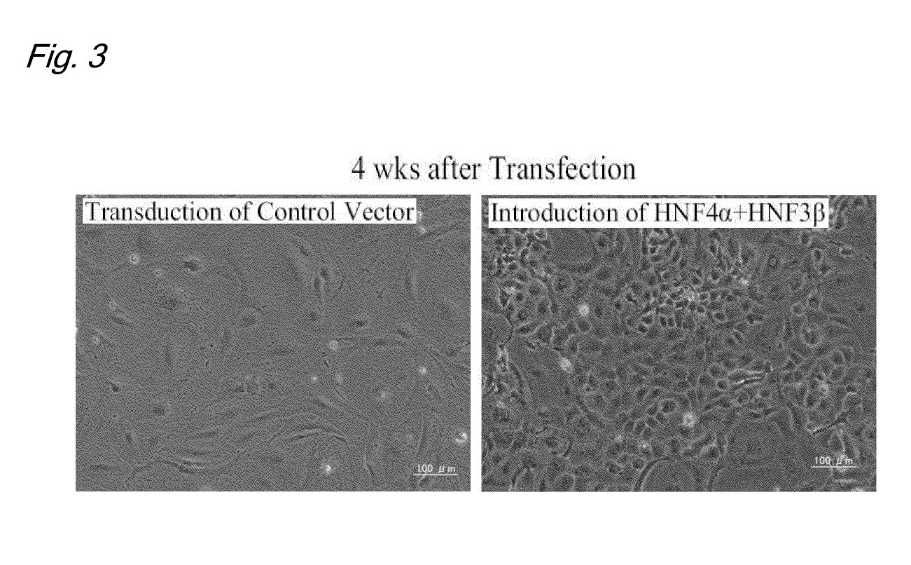

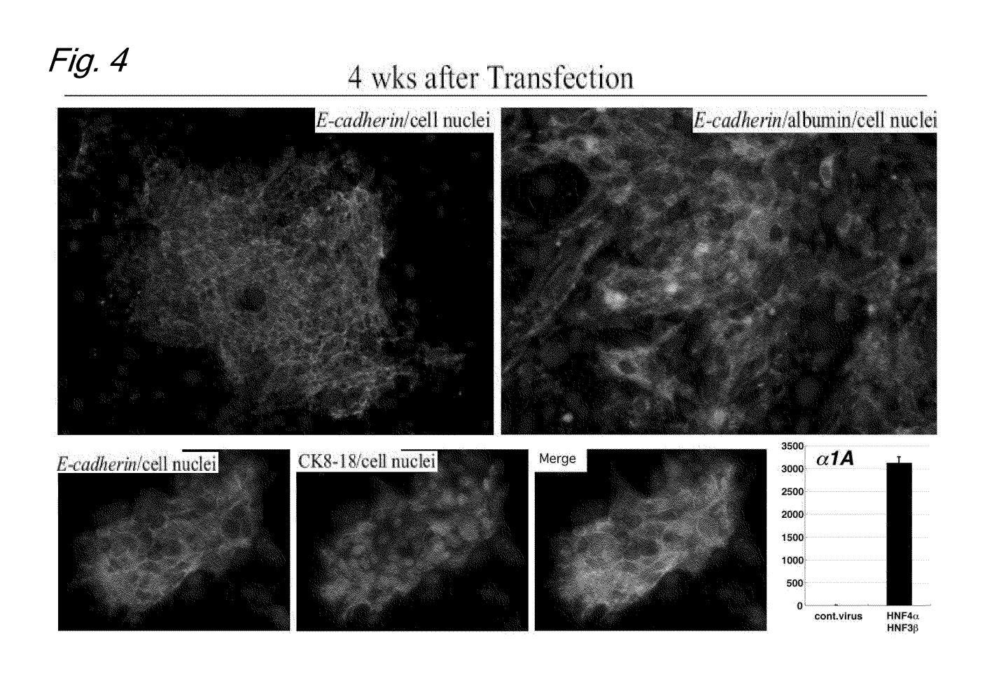

After culturing the MEFs transduced with HNF4α and HNF3β for two weeks, they were transferred into a type I collagen-coated dish and continued to be cultured in a medium containing both HGF and EGF for hepatic stem cells. As a result, it was found that epithelial-like cells totally different in cell morphology from the MEFs had emerged (FIG. 3). The immunostaining of these epithelial-like cells revealed that all of the cells were E-cadherin-positive and also had acquired an epithelial cell-specific morphology characterized in that E-cadherin was localized in cell-adhesion domains (FIG. 4). It was also revealed that many of E-cadherin-positive cells had expressed albumin and cytokeratin 8 and cytokeratin 18 (which are hepatocyte differentiation markers) (FIG. 4). Further, in the induced epithelial-like cells, the expression of α-1-antitrypsin (which is a mid-stage hepatocyte differentiation marker) was observed as well (FIG. 4).

The above experimental results revealed that the epithelial-like cells produced from the MEFs by the transduction of HNF4α, and HNF3β genes were hepatic epithelial cells. The MEFs in culture become senescent in an early stage and undergo cell death, while the newborn epithelial-like cell population continues to proliferate and can be subjected to the subsequent passage culture and cryopreservation. Thus, it is considered that E-cadherin-positive hepatic epithelial cell population includes differentiated hepatocytes and less differentiated hepatic progenitor cells. More specifically, it is supposed that many hepatocytes are supplied from the hepatic progenitor cells having high growth ability, over a long time.

Reconstruction of Liver Tissues by Epithelial-Like Cells Induced from Fibroblasts

To evaluate that the epithelial-like cells induced from the MEFs by the transduction of HNF4α and HNF3β genes were hepatic epithelial cells, thus obtained epithelial-like cells were transplanted into the liver of an FAH knockout mouse and their ability to reconstruct hepatic tissues was analyzed.

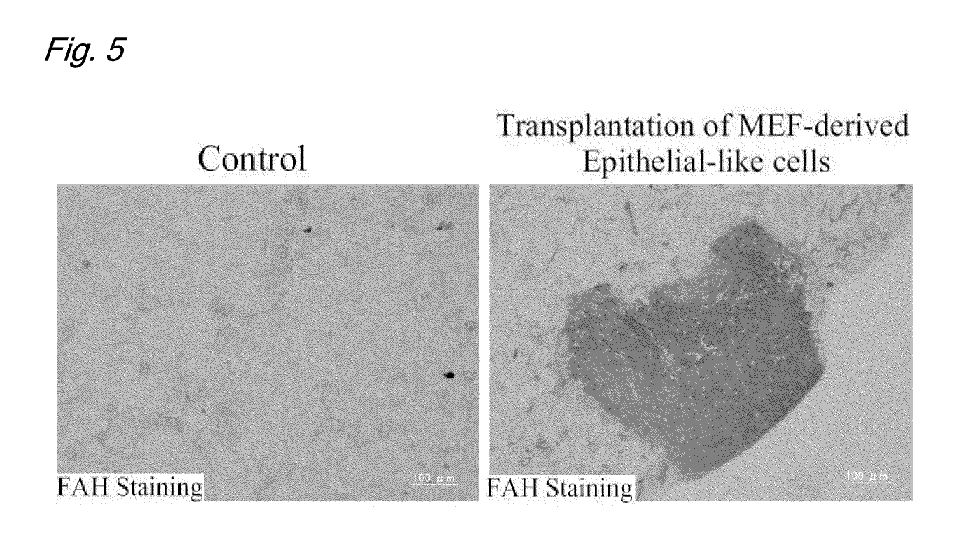

As a result, it was found that one month after the transplantation, the donor cells had been engrafted, as FAH-positive matured hepatocytes, into the hepatic tissues of the FAH knockout mouse and had reconstructed the hepatic tissues (FIG. 5).

The experimental result revealed that the epithelial-like cells induced from the MEFs by the transduction of HNF4α and HNF3β genes were hepatic epithelial cells having the ability to reconstruct hepatic tissues.

### Example 2

Human skin-derived fibroblasts purchased from Cell Applications were cultured in accordance with the protocol attached thereto. The same induction medium and vector as in Example 1 were used. In this way, the gene expression-inducing effect of the transduction of HNF4α and HNF3γ genes, which are shown in the table below, was evaluated.

TABLE 4

| | | | | Corresponding |
|---|---|---|---|---|
| | | | NCBI Accession | Amino Acid |
| | | | No. | Sequence |
| | | | (SEQ ID No. in | (SEQ ID No. in |
| Name | Features | | Sequence Listing) | Sequence Listing) |
| HNF3γ | Transcription factor having a forkhead DNA- | | NM_004497 | (SEQ ID NO: 28) |
| | binding domain. | | | |
| | Involved in the development of endoderm, liver, | | | |
| | and pancreas. | | | |
| HNF4α | Transcription factor | transcript variant 1 | NM_178849 | (SEQ ID NO: 5) |
| | belonging to a nuclear | transcript variant 2 | NM_000457 | (SEQ ID NO: 6) |
| | receptor family. | transcript variant 3 | NM_178850 | (SEQ ID NO: 7) |
| | Involved in hepatocyte | transcript variant 4 | NM_175914 | (SEQ ID NO: 8) |
| | differentiation and cell | transcript variant 5 | NM_001030003 | (SEQ ID NO: 9) |
| | morphology control. | | | |

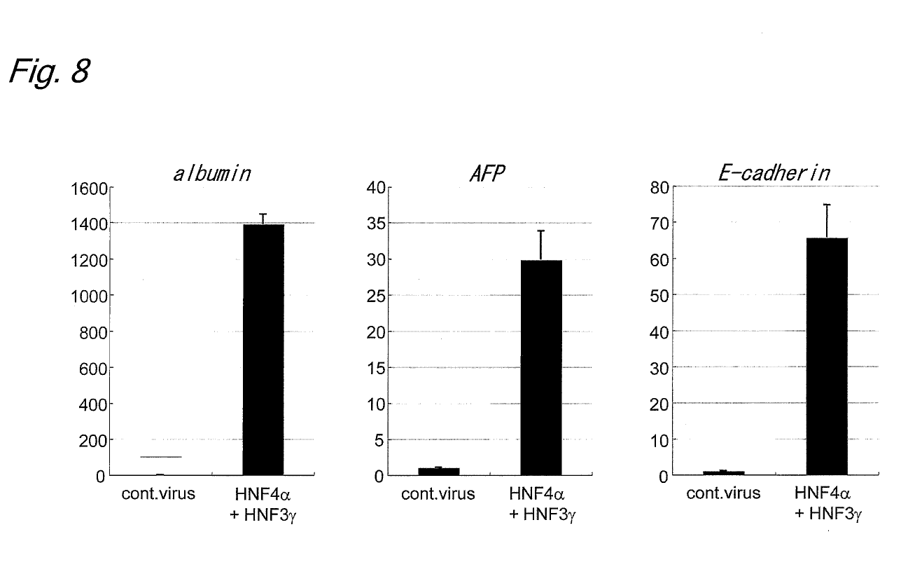

FIG. 8 shows the experimental results obtained by the use of the transcript variant 3.

As a result of the tests for the transcript variants 1 to 5 of HNF4α, it appeared that the transcript variants 1 to 3, especially, the transcript variant 3 was effective. In addition, it was confirmed that, when the human-derived cells were used, gene expression had been induced by the transduction of HNF4α+HNF3α genes or HNF4α+HNF3β genes; however, as in the case of the mouse-derived cells, there was the tendency that the combination of HNF4α and HNF3γ was the most effective.

### DOCUMENTS CITED IN EXAMPLES

Reference 1: Suzuki A., Iwama A., Miyashita H., Nakauchi H., Taniguchi H. Role for growth factors and extracellular matrix in controlling differentiation of prospectively isolated hepatic stem cells. Development 130, 2513-2524, 2003.
Reference 2: Suzuki A., Zheng Y. W., Kondo R., Kusakabe M., Takada Y., Fukao K., Nakauchi H., Taniguchi H. Flow cytometric separation and enrichment of hepatic progenitor cells in the developing mouse liver. Hepatology 32, 1230-1239, 2000.
Reference 3: Suzuki A., Zheng Y. W., Kaneko S., Onodera M., Fukao K., Nakauchi H., Taniguchi H. Clonal identification and characterization of self-renewing pluripotent stem cells in the developing liver. J Cell Biol 156, 173-184, 2002.
Reference 4: Suzuki A., Sekiya S., Onishi M., Oshima N., Kiyonari H., Nakauchi H., Taniguchi H. Flow cytometric isolation and clonal identification of self-renewing bipotent hepatic progenitor cells in adult mouse liver. Hepatology 48, 1964-1978, 2008.
Reference 5: Kaneko S, Onodera M, Fujiki Y, Nagasawa T, Nakauchi H. Simplified retroviral vector gcsap with murine stem cell virus long terminal repeat allows high and continued expression of enhanced green fluorescent protein by human hematopoietic progenitors engrafted in nonobese diabetic/severe combined immunodeficient mice. Hum Gene Ther 12, 35-44, 2001.

1. A method of producing an induced hepatocyte from a non-hepatic cell, comprising the step of introducing the combination of HNF3α, HNF3β, or HNF3γ and HNF4α into a non-hepatic cell.

2. The method of claim 1, comprising the step of transducing, into a non-hepatic cell, the gene encoding HNF3α, HNF3β, or HNF3γ and the gene encoding HNF4α.

3. The method of claim 1, wherein the non-hepatic cell is of human origin.

4. An artificial liver tissue comprising an induced hapatocyte produced by the method of claim 1 or the progeny of the induced hapatocyte.

5. A cell derived from a non-hepatic cell and transduced with the combination of the gene encoding HNF3α, HNF3β, or HNF3γ and the gene encoding HNF4α.

6. A cell induced from a non-hepatic cell transduced with the combination of the gene encoding HNF3α, HNF3β, or HNF3γ and the gene encoding HNF4α, wherein the induced cell being E-cadherin-positive and capable of expressing albumin, cytokeratin-8, cytokeratin-18, or α-1-antitrypsin.

7. A set of factors comprising the combination of HNF3α, HNF3β, or HNF3γ and the gene encoding HNF4α.

8. A set of genes comprising the gene encoding HNF3α, HNF3β, or HNF3γ and the gene encoding HNF4α.

9. A method of producing an induced hepatic cell, comprising the step of culturing, in the presence of a growth factor, a non-hepatic cell transduced with the combination of the gene encoding HNF3α, HNF3β, or HNF3γ and the gene encoding HNF4α.

10. A method of transplanting a cell into liver tissue, comprising the step of transplanting the cell of claim 5.

11. A method of treating diseases, comprising a step using the cell of claim 5.

12. A method of assessing drug responsiveness, comprising a step using the cell of claim 5.

13. The method of claim 2, wherein the non-hepatic cell is of human origin.

14. An artificial liver tissue comprising an induced hapatocyte produced by the method of claim 2 or the progeny of the induced hapatocyte.

15. An artificial liver tissue comprising an induced hapatocyte produced by the method of claim 3 or the progeny of the induced hapatocyte.


SEQUENCE LISTING (28 sequences)

SEQ ID NO: 1 - DNA, 1425 bp, Mouse
  Inventor Suzuki, Atsushi
atgcgactctctaaaacccttgccggcatggatatggccgactacagcgctgccctggacccagcctacaccaccctggagtttgaaaatgtgcaggtgttgaccatgggcaatgacacgtccccatctgaaggtgccaacctcaattcatccaacagcctgggcgtcagtgccctgtgcgccatctgtggcgaccgggccaccggcaaacactacggagcctcgagctgtgacggctgcaaggggttcttcaggaggagcgtgaggaagaaccacatgtactcctgcaggtttagccgacaatgtgtggtagacaaagataagaggaaccagtgtcgttactgcaggcttaagaagtgcttccgggctggcatgaagaaggaagctgtccaaaatgagcgggaccggatcagcacgcggaggtcaagctacgaggacagcagcctgccctccatcaacgcgctcctgcaggcagaggttctgtcccagcagatcacctctcccatctctgggatcaatggcgacattcgggcaaagaagattgccaacatcacagacgtgtgtgagtctatgaaggagcagctgctggtcctggtcgagtgggccaagtacatcccggccttctgcgaactccttctggatgaccaggtggcgctgctcagggcccacgccggtgagcatctgctgcttggagccaccaagaggtccatggtgtttaaggacgtgctgctcctaggcaatgactacatcgtccctcggcactgtccagagctagcggagatgagccgtgtgtccatccgcatcctcgatgagctggtcctgcccttccaagagctgcagattgatgacaatgaatatgcctgcctcaaagccatcatcttctttgatccagatgccaaggggctgagtgacccgggcaagatcaagcggctgcggtcacaggtgcaagtgagcctggaggattacatcaacgaccggcagtacgactctcggggccgctttggagagctgctgctgctgttgcccacgctgcagagcatcacctggcagatgatcgaacagatccagttcatcaagctcttcggcatggccaagattgacaacctgctgcaggagatgcttctcggagggtctgccagtgatgcaccccacacccaccaccccctgcaccctcacctgatgcaagaacacatgggcaccaatgtcattgttgctaacacgatgccctctcacctcagcaatggacagatgtgtgagtggccccgacccagggggcaggcagccactcccgagactccacagccatcaccaccaagtggctcgggatctgaatcctacaagctcctgccaggagccatcaccaccatcgtcaagcctccctctgccattccccagccaacgatcaccaagcaagaagccatctag

SEQ ID NO: 2 - DNA, 1407 bp, Mouse
atgttagggactgtgaagatggaagggcatgagagcaacgactggaacagctactacgcggacacgcaggaggcctactcctctgtccctgtcagcaacatgaactccggcctgggctctatgaactccatgaacacctacatgaccatgaacaccatgaccacgagcggcaacatgaccccggcttccttcaacatgtcctacgccaacacgggcttaggggccggcctgagtcccggtgctgtggctggcatgccaggggcctctgcaggcgccatgaacagcatgactgcggcgggcgtcacggccatgggtacggcgctgagcccgggaggcatgggctccatgggcgcgcagcccgccacctccatgaacggcctgggtccctacgccgccgccatgaacccgtgcatgagtcccatggcgtacgcgccgtccaacctgggccgcagccgcgcggggggcggcggcgacgccaagacattcaagcgcagctaccctcacgccaagccgccttactcctacatctcgctcatcacgatggccatccagcaggcgcccagcaagatgctcacgctgagcgagatctaccagtggatcatggacctcttcccctattaccgccagaaccagcagcgctggcagaactccatccgccactcgctgtccttcaacgattgtttcgtcaaggtggcacgatccccggacaagccaggcaagggctcctactggacgctgcacccggactccggcaacatgttcgagaacggctgctacttgcgccgccaaaagcgcttcaagtgtgagaagcagccgggggccggaggtgggagtgggggcggcggctccaaagggggcccagaaagtcgcaaggacccctcaggcccggggaaccccagcgccgagtcaccccttcaccggggtgtgcacggaaaggctagccagctagagggcgcgccggccccagggcccgccgccagcccccagactctggaccacagcggggccacggcgacagggggcgcttcggagttgaagtctccagcgtcttcatctgcgccccccataagctccgggccaggggcgctggcatctgtacccccctctcacccggctcacggcctggcaccccacgaatctcagctgcatctgaaaggggatccccgctactcctttaatcaccccttctccatcaacaacctcatgtcctcctccgagcaacagcacaagctggacttcaaggcatacgagcaggcgctgcagtactctccttatggcgctaccttgcccgccagtctgccccttggcagcgcctcagtggccacgaggagccccatcgagccctcagccctggagccagcctactaccaaggtgtgtattccagacccgtgctaaatacttcctag

SEQ ID NO: 3 - DNA, 1380 bp, Mouse
atgctgggagccgtgaagatggaagggcacgagccatccgactggagcagctactacgcggagcccgagggctactcttccgtgagcaacatgaacgccggcctggggatgaatggcatgaacacatacatgagcatgtccgcggctgccatgggcggcggttccggcaacatgagcgcgggctccatgaacatgtcatcctatgtgggcgctggaatgagcccgtcgctagctggcatgtccccgggcgccggcgccatggcgggcatgagcggctcagccggggcggccggcgtggcgggcatgggacctcacctgagtccgagtctgagcccgctcgggggacaggcggccggggccatgggtggccttgccccctacgccaacatgaactcgatgagccccatgtacgggcaggccggcctgagccgcgctcgggaccccaagacataccgacgcagctacacacacgccaaacctccctactcgtacatctcgctcatcaccatggccatccagcagagccccaacaagatgctgacgctgagcgagatctatcagtggatcatggacctcttccctttctaccggcagaaccagcagcgctggcagaactccatccgccactctctctccttcaacgactgctttctcaaggtgccccgctcgccagacaagcctggcaagggctccttctggaccctgcacccagactcgggcaacatgttcgagaacggctgctacctgcgccgccagaagcgcttcaagtgtgagaagcaactggcactgaaggaagccgcgggtgcggccagtagcggaggcaagaagaccgctcctgggtcccaggcctctcaggctcagctcggggaggccgcgggctcggcctccgagactccggcgggcaccgagtccccccattccagcgcttctccgtgtcaggagcacaagcgaggtggcctaagcgagctaaagggagcacctgcctctgcgctgagtcctcccgagccggcgccctcgcctgggcagcagcagcaggctgcagcccacctgctgggcccacctcaccacccaggcctgccaccagaggcccacctgaagcccgagcaccattacgccttcaaccaccccttctctatcaacaacctcatgtcgtccgagcagcaacatcaccacagccaccaccaccatcagccccacaaaatggacctcaaggcctacgaacaggtcatgcactacccagggggctatggttcccccatgccaggcagcttggccatgggcccagtcacgaacaaagcgggcctggatgcctcgcccctggctgcagacacttcctactaccaaggagtgtactccaggcctattatgaactcatcctaa

SEQ ID NO: 4 - DNA, 1062 bp, Mouse
atgctgggctcagtgaagatggaggctcatgacctggccgagtggagctactacccggaggcgggcgaggtgtattctccagtgaatcctgtgcccaccatggcccctctcaactcctacatgaccttgaacccactcagctctccctaccctcccggagggcttcaggcctccccactgcctacaggacccctggcacccccagcccccactgcgcccttggggcccaccttcccaagcttgggcactggtggcagcaccggaggcagtgcttccgggtatgtagccccagggcccgggcttgtacatggaaaagagatggcaaaggggtaccggcggccactggcccacgccaaaccaccatattcctacatctctctcataaccatggctattcagcaggctccaggcaagatgctgaccctgagtgaaatctaccaatggatcatggacctcttcccgtactaccgggagaaccagcaacgttggcagaactccatccggcattcgctgtccttcaatgactgcttcgtcaaggtggcacgctccccagacaagccaggcaaaggctcctactgggccttgcatcccagctctgggaacatgtttgagaacggctgctatctccgccggcagaagcgcttcaagctggaggagaaggcaaagaaaggaaacagcgccacatcggccagcaggaatggtactgcggggtcagccacctctgccaccactacagctgccactgcagtcacctccccggctcagccccagcctacgccatctgagcccgaggcccagagtggggatgatgtggggggtctggactgcgcctcacctccttcgtccacaccttatttcagcggcctggagctcccgggggaactaaagttggatgcgccctataacttcaaccaccctttctctatcaacaacctgatgtcagaacagacatcgacaccttccaaactggatgtggggtttgggggctacggggctgagagtggggagcctggagtctactaccagagcctctattcccgctctctgcttaatgcatcctag

SEQ ID NO: 5 - DNA, 1395 bp, Human
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 6 - DNA, 1425 bp, Human
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 7 - DNA, 1254 bp, Human
atgcgactctccaaaaccctcgtcgacatggacatggccgactacagtgctgcactggacccagcctacaccaccctggaatttgagaatgtgcaggtgttgacgatgggcaatgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaa

SEQ ID NO: 8 - DNA, 1359 bp, Human
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtgtgagtggccccgacccaggggacaggcagccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 9 - DNA, 1329 bp, Human
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggagggtcccccagcgatgcaccccatgcccaccaccccctgcaccctcacctgatgcaggaacatatgggaaccaacgtcatcgttgccaacacaatgcccactcacctcagcaacggacagatgtccacccctgagaccccacagccctcaccgccaggtggctcagggtctgagccctataagctcctgccgggagccgtcgccacaatcgtcaagcccctctctgccatcccccagccgaccatcaccaagcaggaagttatctag

SEQ ID NO: 10 - DNA, 1188 bp, Human
atggtcagcgtgaacgcgcccctcggggctccagtggagagttcttacgacacgtccccatcagaaggcaccaacctcaacgcgcccaacagcctgggtgtcagcgccctgtgtgccatctgcggggaccgggccacgggcaaacactacggtgcctcgagctgtgacggctgcaagggcttcttccggaggagcgtgcggaagaaccacatgtactcctgcagatttagccggcagtgcgtggtggacaaagacaagaggaaccagtgccgctactgcaggctcaagaaatgcttccgggctggcatgaagaaggaagccgtccagaatgagcgggaccggatcagcactcgaaggtcaagctatgaggacagcagcctgccctccatcaatgcgctcctgcaggcggaggtcctgtcccgacagatcacctcccccgtctccgggatcaacggcgacattcgggcgaagaagattgccagcatcgcagatgtgtgtgagtccatgaaggagcagctgctggttctcgttgagtgggccaagtacatcccagctttctgcgagctccccctggacgaccaggtggccctgctcagagcccatgctggcgagcacctgctgctcggagccaccaagagatccatggtgttcaaggacgtgctgctcctaggcaatgactacattgtccctcggcactgcccggagctggcggagatgagccgggtgtccatacgcatccttgacgagctggtgctgcccttccaggagctgcagatcgatgacaatgagtatgcctacctcaaagccatcatcttctttgacccagatgccaaggggctgagcgatccagggaagatcaagcggctgcgttcccaggtgcaggtgagcttggaggactacatcaacgaccgccagtatgactcgcgtggccgctttggagagctgctgctgctgctgcccaccttgcagagcatcacctggcagatgatcgagcagatccagttcatcaagctcttcggcatggccaagattgacaacctgttgcaggagatgctgctgggaggtccgtgccaagcccaggaggggcggggttggagtggggactccccaggagacaggcctcacacagtgagctcacccctcagctccttggcttccccactgtgccgctttgggcaagttgcttaa

SEQ ID NO: 11 - DNA, 1419 bp, Human
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 12 - DNA, 1392 bp, Human
atgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaa

SEQ ID NO: 13 - DNA, 1374 bp, Human
atgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaa

SEQ ID NO: 14 - DNA, 1053 bp, Human
atgctgggctcagtgaagatggaggcccatgacctggccgagtggagctactacccggaggcgggcgaggtctactcgccggtgaccccagtgcccaccatggcccccctcaactcctacatgaccctgaatcctctaagctctccctatccccctggggggctccctgcctccccactgccctcaggacccctggcacccccagcacctgcagcccccctggggcccactttcccaggcctgggtgtcagcggtggcagcagcagctccgggtacggggccccgggtcctgggctggtgcacgggaaggagatgccgaaggggtatcggcggcccctggcacacgccaagccaccgtattcctatatctcactcatcaccatggccatccagcaggcgccgggcaagatgctgaccttgagtgaaatctaccagtggatcatggacctcttcccttactaccgggagaatcagcagcgctggcagaactccattcgccactcgctgtctttcaacgactgcttcgtcaaggtggcgcgttccccagacaagcctggcaagggctcctactgggccctacaccccagctcagggaacatgtttgagaatggctgctacctgcgccgccagaaacgcttcaagctggaggagaaggtgaaaaaagggggcagcggggctgccaccaccaccaggaacgggacagggtctgctgcctcgaccaccacccccgcggccacagtcacctccccgccccagcccccgcctccagcccctgagcctgaggcccagggcggggaagatgtgggggctctggactgtggctcacccgcttcctccacaccctatttcactggcctggagctcccaggggagctgaagctggacgcgccctacaacttcaaccaccctttctccatcaacaacctaatgtcagaacagacaccagcacctcccaaactggacgtggggtttgggggctacggggctgaaggtggggagcctggagtctactaccagggcctctattcccgctctttgcttaatgcatcctag

SEQ ID NO: 15 - protein, 474 aa, Mouse
MRLSKTLAGMDMADYSAALDPAYTTLEFENVQVLTMGNDTSPSEGANLNSSNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSQQITSPISGINGDIRAKKIANITDVCESMKEQLLVLVEWAKYIPAFCELLLDDQVALLRAHAGEHLLLGATKRSMVFKDVLLLGNDYIVPRHCPELAEMSRVSIRILDELVLPFQELQIDDNEYACLKAIIFFDPDAKGLSDPGKIKRLRSQVQVSLEDYINDRQYDSRGRFGELLLLLPTLQSITWQMIEQIQFIKLFGMAKIDNLLQEMLLGGSASDAPHTHHPLHPHLMQEHMGTNVIVANTMPSHLSNGQMCEWPRPRGQAATPETPQPSPPSGSGSESYKLLPGAITTIVKPPSAIPQPTITKQEAI

SEQ ID NO: 16 - protein, 468 aa, Mouse
MLGTVKMEGHESNDWNSYYADTQEAYSSVPVSNMNSGLGSMNSMNTYMTMNTMTTSGNMTPASFNMSYANTGLGAGLSPGAVAGMPGASAGAMNSMTAAGVTAMGTALSPGGMGSMGAQPATSMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGSGGGGSKGGPESRKDPSGPGNPSAESPLHRGVHGKASQLEGAPAPGPAASPQTLDHSGATATGGASELKSPASSSAPPISSGPGALASVPPSHPAHGLAPHESQLHLKGDPHYSFNHPFSINNLMSSSEQQHKLDFKAYEQALQYSPYGATLPASLPLGSASVATRSPIEPSALEPAYYQGVYSRPVLNTS

SEQ ID NO: 17 - protein, 459 aa, Mouse
MLGAVKMEGHEPSDWSSYYAEPEGYSSVSNMNAGLGMNGMNTYMSMSAAAMGGGSGNMSAGSMNMSSYVGAGMSPSLAGMSPGAGAMAGMSGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAASSGGKKTAPGSQASQAQLGEAAGSASETPAGTESPHSSASPCQEHKRGGLSELKGAPASALSPPEPAPSPGQQQQAAAHLLGPPHHPGLPPEAHLKPEHHYAFNHPFSINNLMSSEQQHHHSHHHHQPHKMDLKAYEQVMHYPGGYGSPMPGSLAMGPVTNKAGLDASPLAADTSYYQGVYSRPIMNSS

SEQ ID NO: 18 - protein, 353 aa, Mouse
MLGSVKMEAHDLAEWSYYPEAGEVYSPVNPVPTMAPLNSYMTLNPLSSPYPPGGLQASPLPTGPLAPPAPTAPLGPTFPSLGTGGSTGGSASGYVAPGPGLVHGKEMAKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKAKKGNSATSASRNGTAGSATSATTTAATAVTSPAQPQPTPSEPEAQSGDDVGGLDCASPPSSTPYFSGLELPGELKLDAPYNFNHPFSINNLMSEQTSTPSKLDVGFGGYGAESGEPGVYYQSLYSRSLLNAS

SEQ ID NO: 19 - protein, 464 aa, Human
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMSTPETPQPSPPGGSGSEPYKLLPGAVATIVKPLSAIPQPTITKQEVI

SEQ ID NO: 20 - protein, 474 aa, Human
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 21 - protein, 417 aa, Human
MRLSKTLVDMDMADYSAALDPAYTTLEFENVQVLTMGNDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGPCQAQEGRGWSGDSPGDRPHTVSSPLSSLASPLCRFGQVA

SEQ ID NO: 22 - protein, 452 aa, Human
MVSVNAPLGAPVESSYDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMCEWPRPRGQAATPETPQPSPPGGSGSEPYKLLPGAVATIVKPLSAIPQPTITKQEVI

SEQ ID NO: 23 - protein, 442 aa, Human
MVSVNAPLGAPVESSYDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGSPSDAPHAHHPLHPHLMQEHMGTNVIVANTMPTHLSNGQMSTPETPQPSPPGGSGSEPYKLLPGAVATIVKPLSAIPQPTITKQEVI

SEQ ID NO: 24 - protein, 395 aa, Human
MVSVNAPLGAPVESSYDTSPSEGTNLNAPNSLGVSALCAICGDRATGKHYGASSCDGCKGFFRRSVRKNHMYSCRFSRQCVVDKDKRNQCRYCRLKKCFRAGMKKEAVQNERDRISTRRSSYEDSSLPSINALLQAEVLSRQITSPVSGINGDIRAKKIASIADVCESMKEQLLVLVEWAKYIPAFCELPLDDQVALLRAHAGEHLLLGATKRSMVFKDVLLLGNDYIVPRHCPELAEMSRVSIRILDELVLPFQELQIDDNEYAYLKAIIFFDPDAKGLSDPGKIKRLRSQVQVSLEDYINDRQYDSRGRFGELLLLLPTLQSITWQMIEQIQFIKLFGMAKIDNLLQEMLLGGPCQAQEGRGWSGDSPGDRPHTVSSPLSSLASPLCRFGQVA

SEQ ID NO: 25 - protein, 472 aa, Human
MLGTVKMEGHETSDWNSYYADTQEAYSSVPVSNMNSGLGSMNSMNTYMTMNTMTTSGNMTPASFNMSYANPGLGAGLSPGAVAGMPGGSAGAMNSMTAAGVTAMGTALSPSGMGAMGAQQAASMNGLGPYAAAMNPCMSPMAYAPSNLGRSRAGGGGDAKTFKRSYPHAKPPYSYISLITMAIQQAPSKMLTLSEIYQWIMDLFPYYRQNQQRWQNSIRHSLSFNDCFVKVARSPDKPGKGSYWTLHPDSGNMFENGCYLRRQKRFKCEKQPGAGGGGGSGSGGSGAKGGPESRKDPSGASNPSADSPLHRGVHGKTGQLEGAPAPGPAASPQTLDHSGATATGGASELKTPASSTAPPISSGPGALASVPASHPAHGLAPHESQLHLKGDPHYSFNHPFSINNLMSSSEQQHKLDFKAYEQALQYSPYGSTLPASLPLGSASVTTRSPIEPSALEPAYYQGVYSRPVLNTS

SEQ ID NO: 26 - protein, 463 aa, Human
MHSASSMLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 27 - protein, 457 aa, Human
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 28 - protein, 350 aa, Human
MLGSVKMEAHDLAEWSYYPEAGEVYSPVTPVPTMAPLNSYMTLNPLSSPYPPGGLPASPLPSGPLAPPAPAAPLGPTFPGLGVSGGSSSSGYGAPGPGLVHGKEMPKGYRRPLAHAKPPYSYISLITMAIQQAPGKMLTLSEIYQWIMDLFPYYRENQQRWQNSIRHSLSFNDCFVKVARSPDKPGKGSYWALHPSSGNMFENGCYLRRQKRFKLEEKVKKGGSGAATTTRNGTGSAASTTTPAATVTSPPQPPPPAPEPEAQGGEDVGALDCGSPASSTPYFTGLELPGELKLDAPYNFNHPFSINNLMSEQTPAPPKLDVGFGGYGAEGGEPGVYYQGLYSRSLLNAS